(19) United States
(12) Patent Application Publication
(10) Pub. No.: US 2008/0193597 A1
(43) Pub. Date: Aug. 14, 2008

(54) FOOD SUPPLEMENT COMPRISING SPIRULINA AND A PLANT-BASED PRODUCT OF THE ALOE GENUS, AND THE COSMETIC USE THEREOF

(76) Inventor: Philippe Alleon, Inezgane (MA)

Correspondence Address: STITES & HARBISON PLLC, 1199 NORTH FAIRFAX STREET, SUITE 900, ALEXANDRIA, VA, 22314, US

(21) Appl. No.: 12/031,255

(22) Filed: Feb. 14, 2008

(30) Foreign Application Priority Data
Feb. 14, 2007 (MA) 29680
May 14, 2007 (FR) 07 55046

(51) Int. Cl. C12N 1/20; A23L 1/29; A23L 1/28

(52) U.S. Cl. 426/61

(57) ABSTRACT

The invention relates to a composition comprising spirulina or a product derived from spirulina and a plant-based product of the Aloe genus; the use thereof for producing a food supplement and the resulting food supplement.

The invention relates to a food supplement for strengthening natural defences.

The genus Aloe (division: Magnoliophyta, class: Liliopsida, order: Asparagales, family: Asphodelaceae) comprises approximately 400 species of flowering succulent plants which are generally referred to as aloes, the most well known of which is undoubtedly Aloe vera. The majority of aloes have a cluster of wide, thick and spiked leaves in the form of a lance. They often have no stem, the cluster growing directly on the ground.

It is known to use products derived from Aloe vera in cosmetic preparations.

Spirulina are a genus (Arthrospira) of cyanobacteria—previously referred to as cyanophyta or “blue” algae—from the warm, shallow and brackish hot waters of the intertropical belt. Although generally referred to as “spirulina”, these cyanobacteria belong to the genus Arthrospira. The genus Spirulina exists but it refers to other cyanobacteria which are quite different in terms of taxonomy.

These are microscopic photosynthetic algae which grow naturally in the alkaline waters of some lakes of the intertropical zone.

Owing to their exceptional richness in terms of proteins, spirulina have been cultivated in a number of countries since the 1970s: Africa, the Antilles, China, United States, Hawaii, India, Madagascar, Mexico, Ecuador, Thailand. Recently, they have been cultivated in France, in the Cévennes and in the Var.

Spirulina combat malnutrition, under-nourishment and protein deficiencies, such as kwashiorkor. They are a highly assimilable source of iron. Rich in antioxidant molecules (gamma-linolenic acid, phycocyanine, tocopherol, carotene, selenium and zinc), they are also used in cosmetology.

One of the advantages of spirulina is that they grow in waters which are moderately to highly salted, and alkaline, that is to say, under conditions in which the growth of other microorganisms is limited. Consequently, it is possible to quite readily obtain non-contaminated homogeneous cultures over relatively short periods of time.

Another advantage of spirulina is linked to the high yield of the cultures. In aquatic basins which are a few decimetres in depth and which are exposed to the sun, in alkaline water (pH close to 10) which is maintained at a temperature of between 25 and 35° C., the production reaches 10 g/j/m². After harvesting, filtration, draining, washing, then drying, a fine green powder is obtained.

Spirulina have been the subject of exhaustive toxicological studies which have shown that it is completely harmless.

In addition to the advantages which spirulina cultures have in terms of aid for populations which are affected by malnutrition, spirulina are found to be increasingly successful in industrialised countries amongst sportsmen and nutritionists who find an exceptional source of active ingredients in this natural product.

Advantageous contents include in particular:
iron which is particularly bioavailable and therefore directly absorbable,
vitamin B9, or folic acid, which is involved in the fixing of iron and muscular anabolism,
branched amino acids, Leucine, Isoleucine, Valine, which are particularly sought after for the increase of muscular mass,
vitamins B1, B6 and B12 whose role is essential both for preparation and for recovery after exercise,
liposoluble vitamins D, E, A and in particular provitamin A,
beta carotene which eliminates the risks of excessive metering,
metals (in non-toxic proportions): selenium, copper, magnesium, manganese,
enzymes and co-enzymes which are involved in metabolic reactions, including Super Oxide Dismutase (SOD) which is a powerful antioxidant,
polyunsaturated fatty acids omega 3 and omega 6, more specifically linolenic and dihomo-gamma linolenic acids whose presence is essential for the body; and
phycocyanine, the main photosynthetic pigment of spirulina, which also has antioxidant properties.

Amongst these active ingredients, phycocyanine, SOD and the polyunsaturated fatty dihommo-gamma linolenic and gamma linolenic acids are the most sought after. Phycocyanine is a phycobiliprotein of 332 KDa which is close in terms of the structure thereof to human bile pigments which are known for their anti-inflammatory properties. Studies have shown that phycocyanine, in animals, allowed the prevention of the formation of leukotrienes B4 which are highly inflammatory eicosanoid metabolites, and the activity of cyclooxygenase-2 (COX-2) and lipoxygenase, enzymes which are associated with the inflammatory processes [Romay C. et al., Inflamm. Res., 1998, 47(1):36-41].

The international application WO99/15688 discloses a method for cultivating spirulina in order to produce a biomass which is particularly rich in omega 6, polyunsaturated fatty acids, in particular gamma-linolenic acid, and/or sulpholipids.

Surprisingly, the Applicant has now discovered that the association of spirulina and a plant-based product of the Aloe genus allows a level of activity to be achieved which is very much greater than that of spirulina alone, in particular in order to strengthen the natural defences of the body or to combat physical fatigue.

In this manner, according to a first aspect, the present invention relates to a composition which comprises spirulina or a product derived from spirulina and a plant-based product of the Aloe genus.

In the context of the present invention, the term “plant-based product of the Aloe genus” is intended to refer to a product which is derived from a plant of the Aloe genus, for example, a juice, a pulp, an extract, an oil, a lyophilisate, a dehydration product, a zeodration product or a mixture thereof obtained from an organ, a liquid or a ground material which originates directly from a plant of the Aloe genus. Preferably, this derived product is obtained from the leaves.

In the context of the present invention, the term “plant of the Aloe genus” is preferably intended to refer to Aloe vera or Aloe arborescens and, in a particularly preferred manner Aloe arborescens.

Spirulina refers to a blue-green algae which belongs to the phylum Cyanophyta, class: Cyanophyceae, order: Nostocales, family: Oscillatoriaceae, genus: Arthrospira.

In the context of the present invention, the term “product derived from spirulina” is intended to refer to a culture of spirulina which is separated from the culture medium thereof, optionally washed, optionally drained and/or optionally dried, or an extract, a lyophilisate, a dehydration product, a zeodration product or a mixture thereof obtained from a culture of spirulina.

Advantageously, spirulina or the product derived from spirulina is present in the composition according to the invention at a ratio of from approximately 10% to approximately 80%, preferably from approximately 20% to approximately 60% and in particular approximately 50% by weight of the composition.

Also in an advantageous manner, the plant-based product of the Aloe genus is present at a ratio of from approximately 10% to approximately 80%, preferably from approximately 20% to approximately 60%, in particular approximately 50% by weight of the composition.

The compositions according to the invention may comprise, as an additional component, an extract of noni (Morinda citrifolia).

A method for preparing a composition according to the invention is an additional aspect of the present invention.

In a preparation method of this type, spirulina or a product derived from spirulina is mixed with a plant-based product of the Aloe genus.

Another aspect of the present invention is the use of compositions according to the invention for preparing food supplements. Food supplements of this type also form part of the invention.

In order to ensure an optimal effect within the food supplement in which it is incorporated, the composition according to the invention is generally used in the supplement at a ratio of from 1 to 100% by weight, preferably at a ratio of from approximately 5% to approximately 95% by weight, in a particularly preferred manner, at a ratio of from approximately 60% to approximately 90% by weight.

The food supplements according to the invention may comprise, in addition to the compositions according to the invention, one or more carriers which are acceptable in terms of foodstuffs.

The term “carrier which is acceptable in terms of foodstuffs” is intended to refer to a carrier which is suitable for use via the oral route, with no toxicity or undue allergic reaction or the like and which is proportioned at a reasonable risk/benefit ratio.

The food supplements according to the invention may comprise, in addition to the compositions according to the invention, one or more other additives which are acceptable in terms of foodstuffs, preferably selected from the group which is constituted by colourings, preservatives, antioxidants, texture agents, modified starches, sugar substitutes, flavour enhancers, acidifying agents, emulsifiers, thickening agents, stabilising agents and gelling agents.

Such food supplements according to the invention are generally in the form of a pill, tablet, bar, drink, gel, powder or granules.

A person skilled in the art is capable of determining the nature of the additional component(s) to be used in the food supplements according to the invention, taking into account the type of supplement which it is desirable to obtain.

A food supplement according to the invention may be prepared in accordance with any method known to the person skilled in the art. A method of this type may involve shaping the supplement in the form of pills, tablets, etc., in particular by means of extrusion.

Preferably, the food supplement of the present invention is intended for use in human beings, companion animals, in particular dogs or cats, or animals for rearing.

Another aspect of the invention is the use of a food supplement according to the invention in order to strengthen the natural defences of the body.

Another aspect of the invention is the use of a food supplement according to the invention in order to increase the endurance and the resistance of the body.

Another aspect of the invention is the use of a food supplement according to the invention in order to combat physical fatigue.

Another aspect of the invention is the use of a food supplement according to the invention in order to restore brightness to the skin, nails, and/or hair.

Another aspect of the invention is the use of a food supplement according to the invention in order to complement a slimming regime.

Preferably, the food supplement according to the invention is used by those suffering from malnutrition, convalescents, anorexics, pregnant or breast-feeding women, sportsmen and elderly persons.

The following examples illustrate the invention without limiting the scope thereof.

### EXAMPLE 1

### Preparation of a Composition According to the Invention using Fresh Materials

A culture of spirulina which has been harvested, washed and drained is mixed with a leaf juice of Aloe arborescens obtained by grinding leaves, in accordance with the following proportions:

| | |
|---|---|
| Spirulina: | 50% |
| Aloe arborescens | 50% |

The product obtained is a syrup-like liquid.

### EXAMPLE 2

### Preparation of a Composition According to the Invention using Dry Materials

A culture of spirulina which has been harvested, washed, drained and dried is mixed with a dried ground material from the leaves of Aloe arborescens, in accordance with the following proportions:

| | |
|---|---|
| Spirulina: | 80% |
| Aloe arborescens | 20% |

The product obtained is a powder.

1. Composition comprising spirulina or a product derived from spirulina and a plant-based product of the Aloe genus.

2. Composition according to claim 1, characterised in that the plant-based product of the Aloe genus is a leaf juice from a plant of the Aloe genus.

3. Composition according to claim 1, characterised in that the plant of the Aloe genus is Aloe arborescens.

4. Composition according to claim 1, further comprising an extract of noni (Morinda citrifolia).

5. Composition according to claim 1, characterised in that spirulina or the product derived from spirulina is present at a ratio of from approximately 10% to approximately 80% by weight of the composition.

6. Composition according to claim 1, characterised in that the plant-based product of the Aloe genus is present at a ratio of from approximately 10 to approximately 80% by weight of the composition.

7. Food supplement comprising a composition according to claim 1.

8. Method for strengthening the natural defences of the body, comprising the administration of an acceptable amount of a food supplement according to claim 7.
